(19) United States
(12) Patent Application Publication
(10) Pub. No.: US 2015/0105605 A1
(43) Pub. Date: Apr. 16, 2015

(54) RADIOACTIVE GLASS SOURCE IN OPHTHALMIC BRACHYTHERAPY

(71) Applicant: IP LIBERTY VISION CORPORATION, New York, NY (US)

(72) Inventors: Paul T. FINGER, New York, NY (US); Toby Welles, Redding, CT (US)

(73) Assignee: IP LIBERTY VISION CORPORATION, New York, NY (US)

(21) Appl. No.: 14/243,671

(22) Filed: Apr. 2, 2014

Related U.S. Application Data
(60) Provisional application No. 61/891,349, filed on Oct. 15, 2013.

(51) Int. Cl. A61N 5/10

(52) U.S. Cl. CPC A61N 5/1001
USPC 600/3; 29/428

(57) ABSTRACT

An ophthalmic radiation device and method employing a glass radiation-source in which a radioisotope is implemented as either a neutron-activated radioisotope, or radioisotope molecularly bonded to glass or encased in an encasement material.

### CROSS REFERENCE TO RELATED APPLICATIONS

This application claims the benefit of U.S. Ser. No. 61/891,349, filed on Oct. 15, 2013, which is incorporated by reference herein in its entirety.

### FIELD AND BACKGROUND OF THE INVENTION

The present invention relates to the use of glass radiation-sources in ophthalmic brachytherapy.

### BRIEF DESCRIPTION OF THE DRAWINGS

The subject matter regarded as the invention is particularly pointed out and distinctly claimed in the concluding portion of the specification. The invention, in regards to its features, components and their configuration, operation, and advantages are best understood with reference to the following description and accompanying drawings in which:

FIG. 1 is a schematic, perspective view of an unloaded radiation-source holder of the ophthalmic radiation treatment device of FIG. 1, according to an embodiment;

FIG. 2 is a schematic, cross-sectional view of the radiation-source holder of FIG. 2 along section line A-A loaded with a glass radiation-source as shown in FIG. 1, according to an embodiment;

FIG. 2A is a general, schematic perspective view of an ophthalmic radiation treatment device, according to an embodiment;

FIG. 3 is schematic, top view of an unloaded radiation-plaque, according to an embodiment;

FIG. 3A is schematic, top view of a loaded radiation-plaque, according to an embodiment;

FIG. 3B is a schematic, perspective view of a neutron-activated, glass radiation-source, according to an embodiment;

FIG. 4 is a schematic, perspective view of a radioactive, glass radiation-source, implemented a radioisotope encased in a glass encasement, according to an embodiment;

FIG. 5 is a schematic, perspective view of a radioactive, glass radiation-source, implemented as a non-particulate radioisotopes encased in a glass encasement, according to an embodiment;

FIG. 6 is a schematic, cross-sectional view of the glass radiation-source of FIG. 4 implemented as neutron-activated, glass microspheres encased in a glass encasement, according to an embodiment;

FIG. 7 is a schematic, cross-sectional view of the glass radiation-source of FIG. 4 implemented as particulate radioisotope encased in a glass encasement, according to an embodiment;

FIG. 7A is a schematic, top view of glass radiation-source containing multiple radioisotopes, according to an embodiment;

FIGS. 8 and 9 are schematic views of composite, glass radiation-sources having radioisotope and shielding layers, according to embodiments; and

FIG. 10 is a flow chart depicting a method for a non-limiting method for loading a radiation-source into an ophthalmic radiation device.

It will be appreciated that for clarity, elements shown in the figures may not be drawn to scale. Furthermore, where considered appropriate, reference numerals may be repeated among the figures to indicate corresponding elements.

### DETAILED DESCRIPTION OF EMBODIMENTS THE PRESENT INVENTION

In the following detailed description, numerous details are set forth in order to provide a thorough understanding of the invention. However, it will be understood by those skilled in the art that the present invention may be practiced without these specific details and that well-known methods, procedures, and components have not been described in detail to avoid obscuring the present invention.

Embodiments of the present invention are generally directed to an ophthalmic radiation device and, specifically, to embodiments of a glass, radiation-source used in the device.

The following terms will be used out through the document:

“Ophthalmic brachytherapy” refers to the use of radioactive materials in the treatment of, inter alia, sub-retinal neovascularization associated with Age-Related Macular Degeneration (AMG), and malignant and benign ocular tumors.

“Radiation-source”, “source”, “source material”, “radioactive-source”, “radioisotope” all refer to a radioactive material emitting therapeutic radiation.

“Radiation” includes any one or a combination of, inter alia, alpha particles, beta particles, positrons, Auger electrons, gamma-rays, or x-rays.

“Holder” refers to a structure associated with a treatment wand of an ophthalmic radiation device. The holder is configured to support or to contain a glass radiation-source while a practitioner administers a therapeutic quantity of radiation.

“Radiation-source container” refers to radiation-source holders associated with brachytherapy treatment wand, shells associated with plaque radiation treatment, or other placement-related activities associated with ophthalmic brachytherapy.

“Wand”, “treatment wand”, “body of the wand”, or “wand body” refer to an elongated ergonomic structure extending from a handle and supporting the holder at its distal end, according to embodiments. The wand is contoured to provide the optimal access, visibility, and control, and fatigue-preventive ergonomics for the surgeon. In a certain embodiment, the wand is light transmissive whereas in another embodiment the wand is implemented as non-light transmissive.

“Light guide” refers to substantially transparent solid bodies through which light propagation is directed in accordance with the surface geometry of the body.

“Connection configuration” includes plaque eyelets, or flex tabs, or any other structure providing support. It should be appreciated that support structure integral to both the body supported and the body providing the support is also considered a connection configuration.

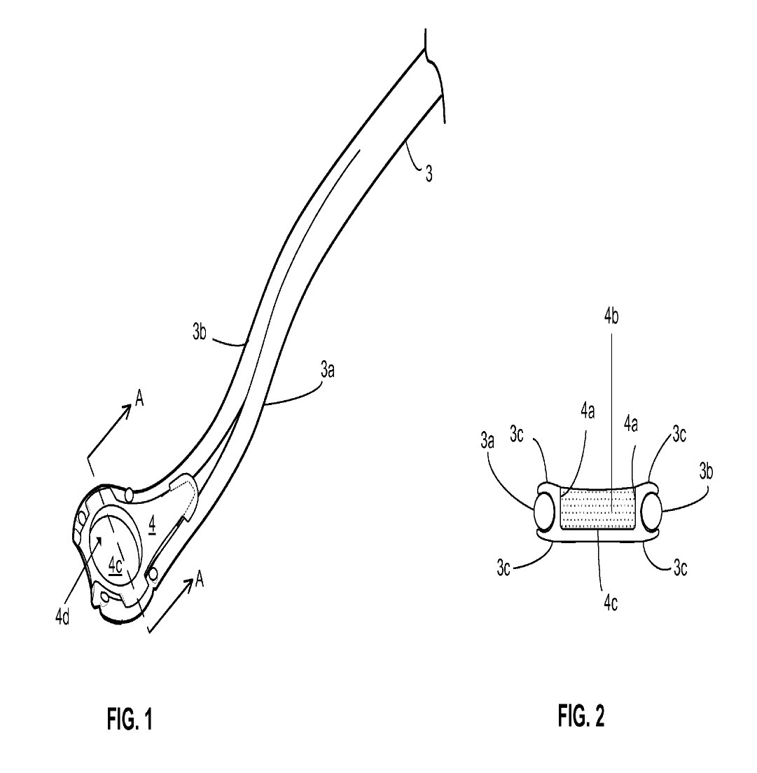

Turning now to the figures, FIGS. 1 and 2 depict a radiation-source container implemented as a holder 4 associated with an ophthalmologic radiation device, in unloaded and loaded states, respectively.

As shown in FIG. 1, holder 4 includes wall 4a (most clearly shown in FIG. 2) and floor 4c that define holding cavity 4d.

In a certain embodiment, holder 4 may include light transmitting elements 3a and 3b of treatment wand 3 as will be further discussed.

Appropriate construction materials of holder 4 include, inter alia, polycarbonate, metal or glass.

FIG. 2 depicts holder 4 loaded with glass radiation-source 4b in holding cavity 4d, shown in FIG. 1. Glass radiation-source 4b may be attached to holder 4 with various degrees of permanence, depending on the embodiment.

In another embodiment, glass radiation-source 4b is permanently connected by way of adhesive or fusion to holder floor 4c or wall 4a. Alternatively, the radioactive source radiation-source 4b may be permanently sealed inside holding cavity 4d with a cover (not shown) fused to wall 4a, or by other retention means.

Permanent connections of glass radiation-source 4b and holder 4 are used in embodiments having either a disposable holder 4, treatment wand 3, or in which the entire ophthalmologic radiation device depicted in FIG. 2A is disposable.

In another embodiment, glass radiation-source 4b is temporally connected by way of adhesive, or corresponding threading embedded in an outer surface of source 4b and wall 4a or to floor 4c, or by way of a removable cover (not shown).

FIG. 2A depicts an ophthalmologic radiation device 2A including a handle 2 connected to treatment wand 3 supporting holder 4, according to an embodiment.

In a certain embodiment, ophthalmologic radiation device 2A includes handle connection configuration 3A providing releasable connection of treatment wand 3 to handle 2, while in an alternative embodiment, holder 4 is releasably connectable to treatment wand 3 via flex tabs 3c as shown in FIG. 2, or alternative connection configurations providing similar functionality. It should be appreciated that non-releasable connection configurations are also included within the scope of the invention.

In some embodiments, ophthalmologic radiation device 2A is fitted with a light pipe 6 for providing light that is transmitted through handle 2 and light transmitting elements 3a and 3b of treatment wand 3 as shown in FIG. 1.

Light transmitting embodiments 3a and 3b may be constructed of strong, substantially transparent polymeric material such as polycarbonate, polysulfone, or polyetherimide, or other material providing sufficient strength and transparency enabling light to propagate through wand 3.

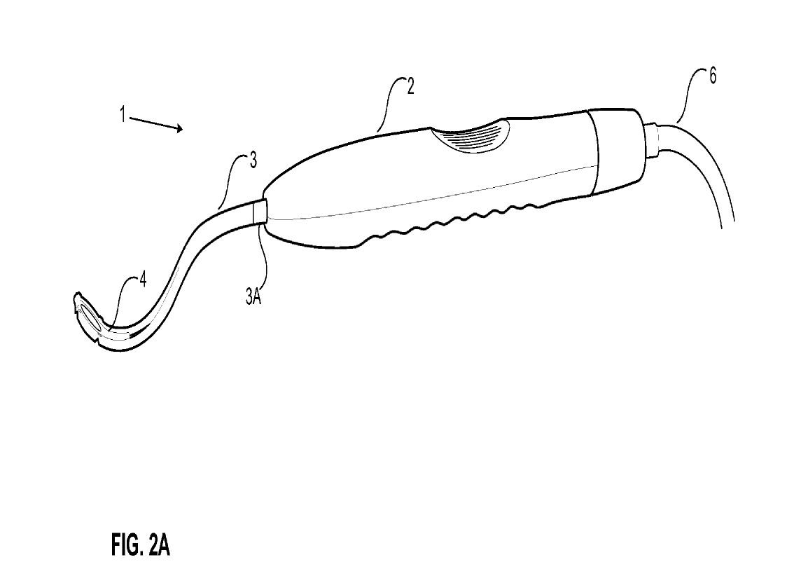

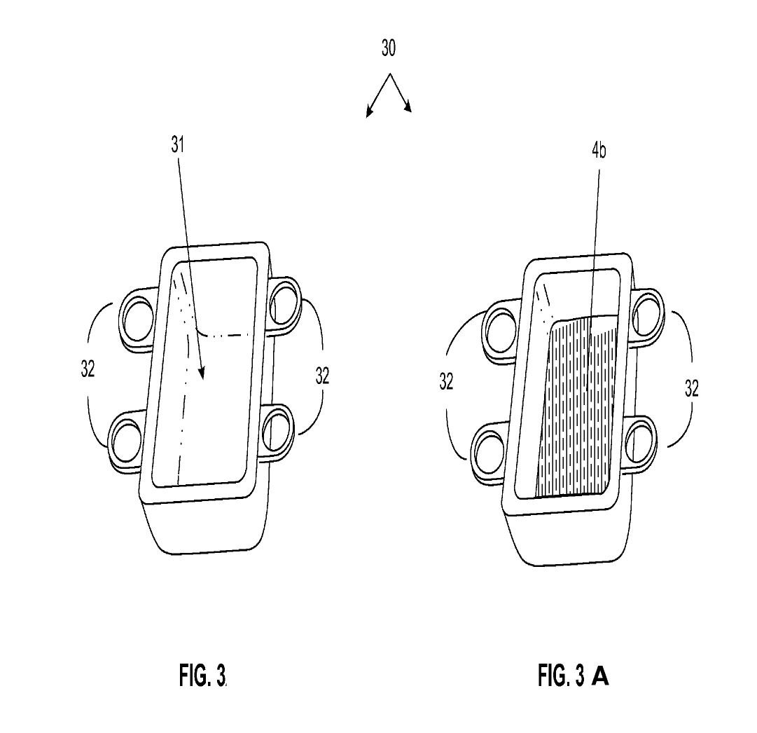

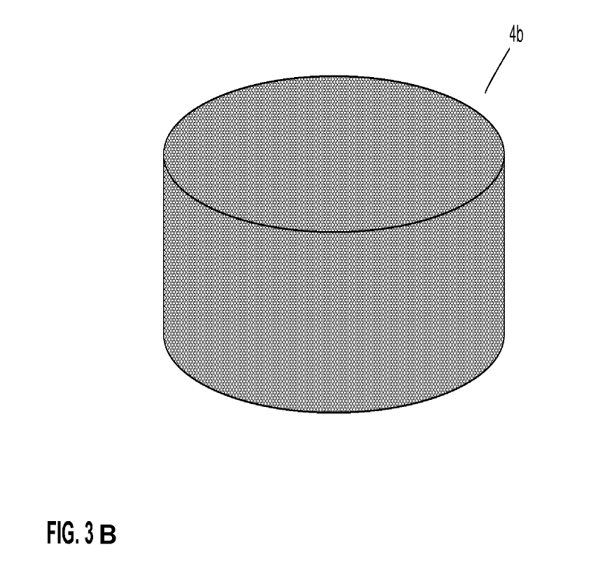

FIGS. 3 and 3A depict a radiation-source container implemented as a radiation plaque 30 or shell having a cavity 31 for receiving a glass radiation-source and eyelets 32 connected to the shell edge so as to enable suture attachment around a treatment area. Plaque 30 may be constructed from materials like, inter alia, gold, silver, steel, and polycarbonate. It should be appreciate that plaque 30 and any corresponding source 4b as shown in FIG. 3A may be constructed to substantially match the contour of a treatment area.

FIG. 3A depicts plaque 30 loaded with glass radiation-source 4b; various options of to which will be discussed.

FIG. 3B depicts a generally cylindrical, glass radiation-source 4b or disk having a thickness of approximately 0.2 mm to 5.0 mm thick and diameter between about 2.0 mm to 22.0 mm, according to an embodiment. It should be appreciated that glass radiation-source 4b can be formed into symmetrical or asymmetrical shapes of assorted surface geometries so as to modulate the radiation field in accordance with a particular need. For example, in certain embodiments a disk-shaped radiation-source may be implemented with a surface substantially matching the curvature of the eye globe.

Without diminishing in scope, a disk-shaped radiation-source will be discussed in this document.

In certain embodiments, radioactive source 4b is implemented as neutron-activated radioisotopes activated through bombardment in a cyclotron with high-energy particles. Such materials include, inter alia, yttrium aluminosilicate, magnesium aluminosilicate, holmium-166, erbium-169, dysprosium-165, rhenium-186, rhenium-188, yttrium-90, or other elements on the periodic table. It should be appreciated that activation through bombardment of particles other than neutrons is also included within the scope of the present invention.

In certain embodiments, non-radioactive glass forming materials are molecularly bonded with a radioactive material. Examples of radioactive materials that may be mixed together or chemically bonded to the glass include, inter alia, iodine-125, palladium-103, and strontium-90 to emit low energy gamma rays.

In certain embodiments, glass radiation-source 4b contains radioisotopes that emit any one or a combination of, inter alia, alpha particles, beta minus and beta plus particles, positrons, Auger electrons, gamma-rays, or x-rays.

The choice of a particular radioisotope or plurality of radioisotopes is defined by the particular therapeutic requirements.

During manufacture, image data of a treatment area maybe derived from data provided by three dimensional medical imaging techniques like, inter alia Magnetic Resonance Imaging (MRI), Three-Dimensional Ultrasound, Computed Axial Tomography (CAT or CT), Single-Photon Emission Computed Tomography (SPECT) or Positron Emission Tomography (PET), for example.

The image includes both surface geometry and shape data that can be used in a variety of manufacturing processes like, inter alia cutting, three-dimensional printing, or other rapid prototyping techniques like laser sintering, stereolithography, or fused filament fabrication. It should be appreciated that in a certain embodiment, these processes may be used to produce a mold for casting or forming glass radiation-source 4b.

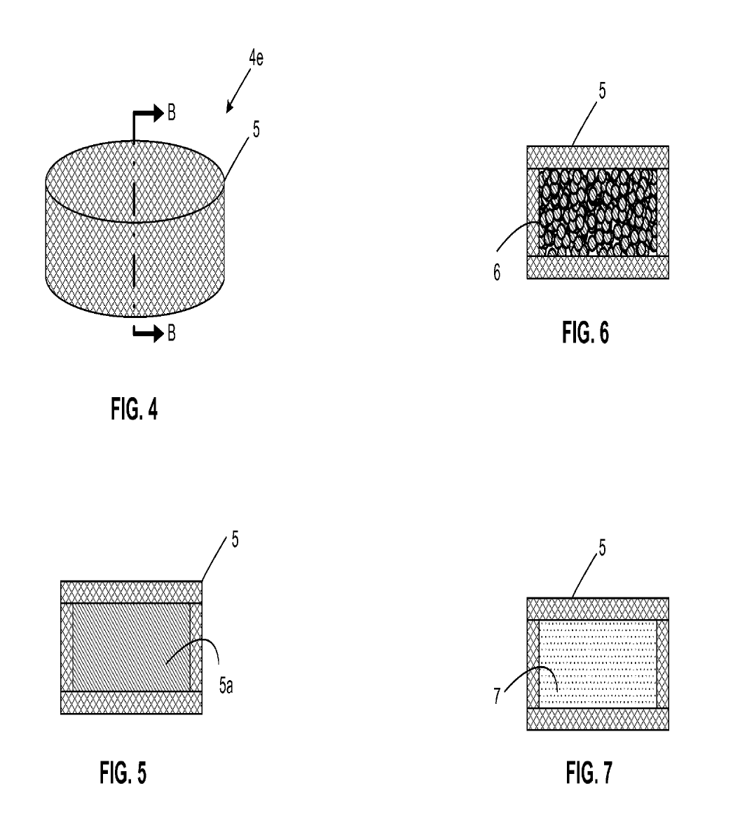

FIG. 4 depicts an embodiment of a radioactive, glass disk 4e implemented as a radioisotope encased in a glass encasement 5 as most clearly seen in the cross-sectional views along line B-B in FIGS. 5-7.

Glass encasement 5 is constructed from silica in certain embodiments; however, it should be appreciated that the glass encasement may be formed from any one or the combination of glass forming oxides including, inter alia, Aluminum Oxide, Boric Oxide, Barium Oxide, Calcium Oxide, Potassium Oxide, Lithium Oxide, Magnesium Oxide, Sodium Oxide, Lead Oxide, Tin Oxide, Strontium Oxide, Zinc Oxide, Titanium Dioxide, and Zirconium Oxide.

In certain embodiments, encasement may be constructed from a radiation-permeable coating of metallic or polymeric material to advantageously contain ablation, fragmentation, detachment, degradation, and selective attenuation of radiation emission.

Glass encasement 5 is formed by any one or a combination of manufacturing processes including, inter cilia, lamination, casting, drawing, forming, molding, blowing, adhesion, or extrusion.

FIG. 5 depicts an embodiment of radioisotope 5a encased inside of a glass encasement 5. Radioisotope 5a may be selected from any one or a combination of, inter alia, ⁸⁹Sr, ¹⁶⁹Yb, ³²P, ³³P, ⁹⁰Y, ¹⁹²Ir, ²⁵I, ¹³¹I, ¹⁰³Pd, ¹⁷⁷Lu, ¹⁴⁹Pm, ¹⁴⁰La, ¹⁵³Sm, ¹⁸⁶Re,

¹⁸⁸Re, ¹⁶⁶Ho, ¹⁶⁶Dy, ¹³⁷Cs, ⁵⁷Co, ¹⁶⁹Er, ¹⁶⁵Dy, ⁹⁷Ru, ^{193m}Pt, ^{195m}Pt, ¹⁰⁵Rh,

⁶⁸Ni, ⁶⁷Cu, ⁶⁴Cu, ¹⁰⁹Cd, ¹¹¹Ag, ¹⁹⁸Au, ¹⁹⁹Au, ²⁰¹Tl, ¹⁷⁵Yb, ⁴⁷Sc, ¹⁵⁹Gd, ²¹²Bi, and ⁷⁷As.

In certain embodiments, of radioisotope 5a is implemented as glass-encased Auger emitters like, inter alia, 67Ga, 99mTc, 111In, 123I, 125I, and 201Tl.

In certain other embodiments, of radioisotope 5a is implemented as glass-encased alpha-emitters like, inter alia, uranium, thorium, actinium, and radium, and the transuranic elements.

FIG. 6 depicts an embodiment of the encased radioisotope 4e of FIG. 4 in which the radioisotope is implemented as microspheres of neutron-activated glass 6 encased in glass encasement 5.

Radioactive microspheres 6 may be implemented from the materials noted above and have an average diameter ranging between 0.2-10.0, according to embodiments.

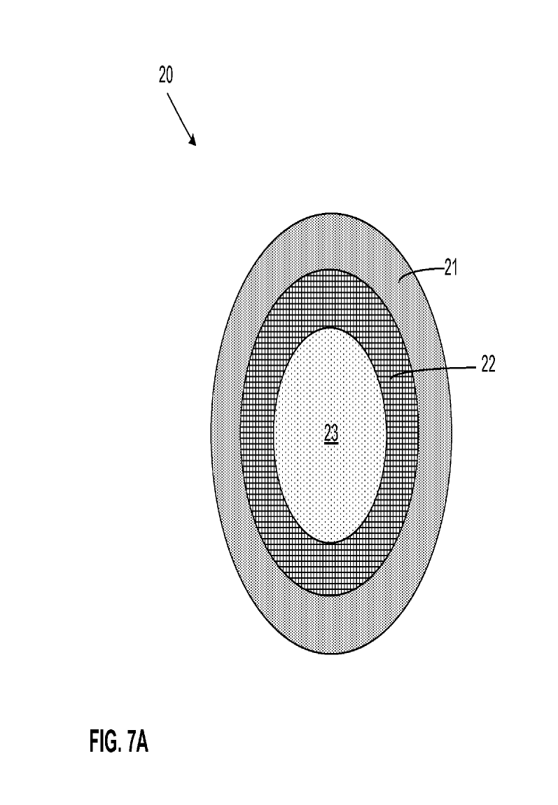

FIG. 7 depicts an embodiment of radioactive glass disk 4e of FIG. 4 in which the radioisotope is implemented as a particulate form of the above noted radioisotopes.

FIG. 7A is a schematic, top view of a glass radiation-source 20 containing multiple radioisotopes 21-23, configured to shape an intraocular dose distribution, according to an embodiment. It should be appreciated that various numbers of differing radioisotopes, or identical isotopes of various concentrations, configured non-concentrically are all included within the scope of the present invention. The various geometrical configurations advantageously facilitate selective exposure to various portions of a treatment area; e.g. a first portion receives a particular exposure, whereas second portion receives a different exposure.

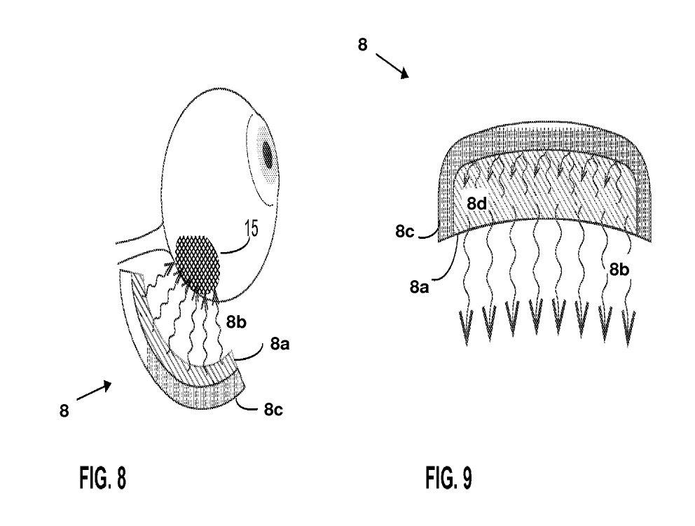

FIGS. 8 and 9 depict a radioactive source 8 implemented as a composite radiation-source having a glass, radioisotope layer 8a and shielding material layer 8c.

FIG. 8 depicts shielding capacity of the composite radiation-source 8. As shown, radioisotope layer 8a directs radiation 8b towards treatment area 15 while shielding layer 8c simultaneously contains or reduces outward, radial radiation. Suitable shielding materials include heavy metals like, inter alia, gold, platinum, steel, tungsten, lead or non-metallic materials like polymeric materials or fluids; the particular material chosen in accordance with the radiation type being shielded.

FIG. 9 depicts reflective capacity of shielding layer 8c of the composite radiation-source 8. As shown, radioisotope layer 8a directs radiation 8b towards treatment area and shielding layer 8c is used to shape the radiation distribution 8d towards the target area thereby enhancing the administration of therapeutic radiation, according to an embodiment.

It should be appreciated that in many embodiments composite, glass-radiation-sources and shielding material inherently shield and reflect simultaneously. Construction methods described above may also be employed to construct composite glass, radiation-sources.

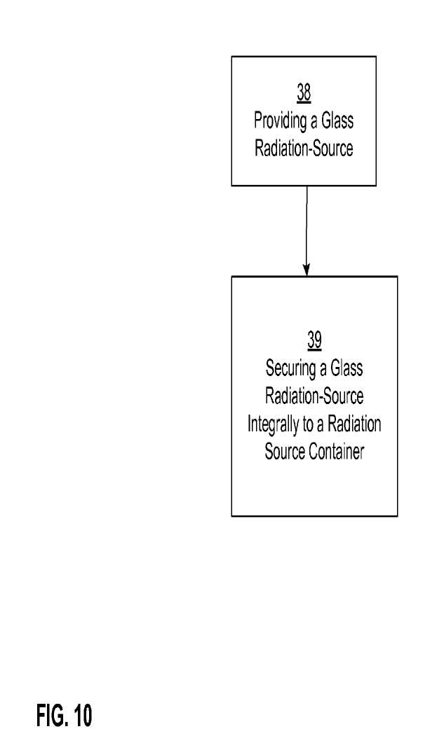

FIG. 10 is a flow chart for a non-limiting method for loading a radiation-source into an ophthalmic radiation device. Specially, in step 38, a glass radiation-source is provided and in step 39 and the glass radiation-source integrally secured to a radiation-source.

It should be appreciated that any combination of the various features and methods are also included within the scope of the invention.

While certain features of the invention have been illustrated and described herein, many to modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An ophthalmic radiation device for delivering a therapeutic dose of radiation to diseased ocular tissue, the radiation device comprising:
a radiation-source container having a plurality of connection configurations enabling support of the container; and
a glass radiation-source disposed in the container.

2. The ophthalmic radiation device of claim 1, wherein the glass radiation-source has a surface boundary substantially matching that of a treatment area so as to minimize radiation in non-treatment area.

3. The ophthalmic radiation device of claim 1, wherein the radiation-source includes radiation emitters selected from the group consisting of emitters of alpha particle, emitters of beta minus and beta plus particles, emitters of Auger electrons, emitters of gamma-rays, and emitters of x-rays.

4. The ophthalmic radiation device of claim 1, wherein the glass radiation-source is implemented as neutron-activated glass selected from the group consisting of yttrium aluminosilicate, magnesium aluminosilicate holmium-166, erbium-169, dysprosium-165, rhenium-186, rhenium-188, and yttrium-90.

5. The ophthalmic radiation device of claim 1, wherein the glass radiation-source is implemented as a radioisotope at least partially encased in an encasement, the encasement constructed from material selected from the group consisting of glass forming material, metallic material, and polymeric material.

6. The ophthalmic radiation device of claim 5, wherein the radioisotope is selected from the group consisting of ⁸⁹Sr, ¹⁶⁹Yb, ³²P, ³³P, ⁹⁰Y, ¹⁹²Ir,
²⁵I, ¹³¹I, ¹⁰³Pd, ¹⁷⁷Lu, ¹⁴⁹Pm, ¹⁴⁰La, ¹⁵³Sm, ¹⁸⁶Re, ¹⁸⁸Re, ¹⁶⁶Ho, ¹⁶⁶Dy,
¹³⁷Cs, ⁵⁷Co, ¹⁶⁹Er, ¹⁶⁵Dy, ⁹⁷Ru, ^{193m}Pt, ^{195m}Pt, ¹⁰⁵Rh, ⁶⁸Ni, ⁶⁷Cu, ⁶⁴Cu,
¹⁰⁹Cd, ¹¹¹Ag, ¹⁹⁸Au, ¹⁹⁹Au, ²⁰¹Tl, ¹⁷⁵Yb, ⁴⁷Sc, ¹⁵⁹Gd, ²¹²Bi, and ⁷⁷As.

7. The ophthalmic radiation device of claim 6, wherein the radioisotope is implemented as a particulate.

8. The ophthalmic radiation device of claim 6, wherein the radioisotope includes neutron-activated glass selected from the group consisting of yttrium aluminosilicate, magnesium aluminosilicate holmium-166, erbium-169, dysprosium-165, rhenium-186, rhenium-188, and yttrium-90.

9. The ophthalmic radiation device of claim 1, wherein the glass radiation-source includes a plurality of radioisotope types.

10. The ophthalmic radiation device of claim 9, wherein each of the plurality of radioisotope types is disposed concentrically.

11. The ophthalmic radiation device of claim 1, wherein the radiation-source container includes a radiation plaque.

12. The ophthalmic radiation device of claim 1, wherein the radiation-source container includes a radiation-source holder associated with a treatment wand.

13. A method for loading a radiation-source into an ophthalmic radiation device, the method comprising:
providing a glass radiation-source having a plurality of connection configurations enabling support of the container; and
securing the glass radiation-source integrally to a radiation-source container.

14. The method of claim 13, wherein the glass radiation-source is implemented as neutron-activated glass, the glass selected from the group of materials consisting of yttrium aluminosilicate, magnesium aluminosilicateholmium-166, erbium-169, dysprosium-165, rhenium-186, rhenium-188, and yttrium-90.

15. The method of claim 13, wherein the glass radiation-source is implemented as a radioisotope at least partially encased in an encasement.

16. The method of claim 15, wherein the encasement is at least partially constructed from one or more materials selected from the group consisting of glass forming material, metallic material, and polymeric material.

17. The method of claim 13, wherein the glass radiation-source includes a radioisotope selected from the group consisting of ⁸⁹Sr, ¹⁶⁹Yb, ³²P, ³³P, ⁹⁰Y, ¹⁹²Ir,
²⁵I, ¹³¹I, ¹⁰³Pd, ¹⁷⁷Lu, ¹⁴⁹Pm, ¹⁴⁰La, ¹⁵³Sm,
¹⁸⁶Re, ¹⁸⁸Re, ¹⁶⁶Ho, ¹⁶⁶Dy, ¹³⁷Cs, ⁵⁷Co, ¹⁶⁹Er, ¹⁶⁵Dy, ⁹⁷Ru, ^{193m}Pt, ^{195m}Pt,
¹⁰⁵Rh, ⁶⁸Ni, ⁶⁷Cu, ⁶⁴Cu, ¹⁰⁹Cd, ¹¹¹Ag, ¹⁹⁸Au, ¹⁹⁹Au, ²⁰¹Tl, ¹⁷⁵Yb, ⁴⁷Sc,
¹⁵⁹Gd ²¹²Bi, and ⁷⁷As.

18. The method of claim 13, wherein the glass radiation-source includes a plurality of radioisotope types or a plurality of radioisotope concentrations.

19. The method of claim 18, wherein the plurality of radiation types are configured to direct a first radiation type into a first portion of a treatment area and a second radiation type into a second portion of the treatment area.

20. The method of claim 18, wherein the plurality of radiation concentrations are configured to direct a first radiation concentration into a first portion of a treatment area and a second radiation concentration into a second portion of the treatment area.

21. The method of claim 18, wherein each of the plurality of radioisotope types or each of the plurality of the radiation concentrations are disposed concentrically.

22. The method of claim 13, wherein the radiation-source container includes a radiation plaque.

23. The method of claim 13, wherein the radiation-source container includes a radiation-source holder associated with a treatment wand.
